(19) United States
(12) Patent Application Publication
(10) Pub. No.: US 2015/0352196 A1
(43) Pub. Date: Dec. 10, 2015

(54) Methods and Pharmaceutical Compositions for Prevention of Aging-Related Lesions

(71) Applicants: INSERM (INSTITUT NATIONAL DE LA SANTÉ ET DE LA RECHERCHE MÉDICALE), Paris (FR); ASSISTANCE PUBLIQUE-HÔPITAUX DE PARIS, Paris (FR)

(72) Inventors: Emmanuel Letavernier, Paris (FR); Laurent Baud, Paris (FR)

(21) Appl. No.: 14/653,677

(22) PCT Filed: Dec. 18, 2013
(86) PCT No.: PCT/EP2013/077216

§ 371 Date: Jun. 18, 2015

(30) Foreign Application Priority Data
Dec. 19, 2012 (EP) 12306618.5

(51) Int. Cl. A61K 38/57

(52) U.S. Cl. CPC A61K 38/57

(57) ABSTRACT

The present invention relates to methods and compositions for prevention of aging-related lesions. More particularly, the present invention relates to a calpain inhibitor compound for use in a method for preventing aging-related lesions in a subject in need thereof.

### FIELD OF THE INVENTION

The present invention relates to methods and compositions for the prevention of aging-related lesions.

### BACKGROUND OF THE INVENTION

The calpain isoforms 1 and 2 (or μ and m) are ubiquitous intracellular proteases activated by micro and millimolar calcium concentrations in vitro, respectively. They have a huge number of substrates and promote cytoskeleton remodeling and cell motility, onset of inflammation, and may induce cell apoptosis (1, 2). Their natural and ubiquitous inhibitor is calpastatin, whose compartimentalization within cells at rest differs from calpains. When calpains are activated, calpastatin co-segregates with them as a pseudo-substrate, limiting by this way their activity.

It has been shown previously that calpain activity increases and calpastatin cellular content decreases with age in mammal brain (3). Calpains promote directly Tau protein accumulation in neurons and therefore participate in Alzheimer disease lesions (4). The administration of pharmacological calpain inhibitors improves cerebral performances and overexpression of calpastatin decreases pathological lesions in murine Alzheimer models (5). Calpain activity is however not limited to brain: it has been shown that calpains promote cataractogenesis and they may be involved downstream of angiotensin II signaling in old animals (6, 7).

It was previously shown that mice overexpressing calpastatin, limiting therefore an overhelming calpain activity in critical conditions, are protected against cardiovascular and kidney lesions induced by chronic angiotensin II infusion, although blood pressure increases similarly in controls and transgenic animals (1). In vitro experiments evidence that angiotensin II activates calpains in smooth muscle cells by AT-1 receptor signalling (1, 7).

Although calpain activity correlates with aging, there is currently no data showing that specific calpain inhibition by genetic or pharmacological ways protects tissues against aging.

### SUMMARY OF THE INVENTION

The present invention relates to a calpain inhibitor compound for use in a method for preventing aging-related lesions in a subject in need thereof.

### DETAILED DESCRIPTION OF THE INVENTION

The role of calpain inhibitors in aging was investigated by the inventors in 2 years-old transgenic mice overexpressing calpastatin and in several cell lines expressing IGF-1, a receptor playing an important role in aging process. The inventors surprisingly found that specific calpain inhibition protects arteries, kidneys and skin and hair against aging-related lesions. The inventors also demonstrate that specific calpain inhibition protects against telomeres shortening and aging lesions in various tissues including kidneys, arteries and skin. Thus, there is for the first time the demonstration that specific calpain inhibition was able to prevent cutaneous, vascular and kidney aging process. Skin of old mice with decreased calpain activity remained similar to young mice skin. Inventors also demonstrate that IGF-1 was able to increase calpain activity in a dose- and time-dependent manner in cell lines, including immortalized kidney cells (podocytes), providing evidence that somatotropic axis, which is implied in aging process, results in calpain activation.

The inventors also assessed skin senescence in 2-months and 2-years old female mice at three levels: (i) Phenotypic evidence, (ii) Histopathological modifications, especially lipofuscin deposits, and (iii) Measure of telomere length in skin. The inventors demonstrated the role of calpain inhibition as protecting against aging-related lesions in skin. At 2 years, all of the 10 control animals exhibited discoloration of the fur and sparse depilation whereas none of the calpastatin-overexpressing mice had alterations of fur. Lipofuscin deposits observed by autofluorescence were abundant in most of 2-years old control mice but rare in 2-years old calpastatin transgenic animals. Telomere shortening was significant in control mice between 2 months and 2 years but not significant between 2-months and 2-years old transgenic calpastatin mice, proving therefore that calpastatin over-expression protects against skin aging.

### Therapeutic Methods and Uses

Accordingly the present invention relates to a calpain inhibitor compound for use in a method for preventing aging-related lesions in a subject in need thereof.

As used herein, the term “subject” denotes a mammal. In a preferred embodiment of the invention, a subject according to the invention refers to any subject (preferably human) susceptible of having aging-related lesions.

As used herein, the term “aging-related lesions” has its general meaning in the art and refers to cellular and functional alterations of a tissue or an organ that progress with aging. Said alterations often lead to excessive repairing mechanisms such as fibrosis. In particular, aging-related lesions are characterized at cellular level by oxydative stress, lipofuscin accumulation, and telomere shortening.

The method of the invention may be performed for any type of aging-related lesions selected from the group consisting of cutaneous aging-related lesions, hair aging-related lesions, vascular aging-related lesions, brain aging-related lesions, heart aging-related lesions and kidney aging-related lesions.

In particular, calpain inhibitors of the invention are particularly suitable for preventing cutaneous anf hair aging-related lesions.

Hair aging-related lesions include but are not limited to alopecia, depilation, whitening or graying of the hair.

Cutaneous aging-related lesions include but are not limited to dermatitis, wrinkling, laxity, sagging, fragility of the skin, dermal matrix alterations, and skin atrophy.

In particular, cutaneous aging-related lesions lead to a number of physiological changes including dryness, roughness, delayed healing and uneven pigmentation, increased photocarcinogenesis, malignancies, fragmentation of collagen and elastic fibers, wrinkles and lax skin, increased risk of pressure damage and decubitus ulcers, purpuric lesions, increased tendencies to injuries, reduced hair growth and effects of androgen, reduced function of sebaceous glands, delayed healing and vulnerability to infection, reduced subcutaneous fat, flattening of dermal papillae, reduced nail growth, decreased linear growth, onychogryphosis, longitudinal striations, dull and brittle nails, decrease in melanocytes, gray hair, and increased susceptibility to solar radiation. Accordingly, calpain inhibitors of the invention are particularly suitable for the prevention of said physiological changes.

The term “calpain” has its general meaning in the art and refers to Ca²⁺-dependent cysteine proteases expressed in all mammalian systems. Calpain 1 (μ-calpain) and calpain 2 (m-calpain) are the most widely distributed calpain isoforms in mammalian cells and are referred to as conventional calpains (Donkor, 2011).

The term “calpastatin” has its general meaning in the art and refers to an endogenous protease inhibitor that acts specifically on calpain. Calpastatin consists of four repetitive sequences of 120 to 140 amino acid residues (domain I, II, III and IV), and a non-inhibitory N-terminal sequence (domain L) (Donkor, 2011).

The term “calpain inhibitor compound” refers to any compound able to prevent the action of calpain. The calpain inhibitor compound of the present invention is a compound that inhibits or reduces the activity of calpain. However, decreasing and/or reducing the activity of calpain can also be obtained by inhibiting calpain expression. Calpain inhibitor compounds of the present invention may consist but are not limited to inhibitor of calpain activity, calpain expression inhibitor, calpastatin activator or calpastatin expression activator.

The term “expression” when used in the context of expression of a gene or nucleic acid refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include messenger RNAs which are modified, by processes such as capping, polyadenylation, methylation, and editing, and proteins (e.g., calpain or calpastatin) modified by, for example, methylation, acetylation, phosphorylation, ubiquitination, SUMOylation, ADP-ribosylation, myristilation, and glycosylation.

An “activator of expression” refers to a natural or synthetic compound that has a biological effect to activate the expression of a gene.

An “inhibitor of expression” refers to a natural or synthetic compound that has a biological effect to inhibit the expression of a gene.

In one embodiment, the calpain inhibitor compound of the invention is an inhibitor of calpain activity. Said inhibitor of calpain activity may be selected from the group consisting of small organic molecules, peptides, aptamers or antibodies (preferably intra-antibodies).

The inhibitors of calpain activity are well-known in the art as illustrated by Donkor, (2011), Pietch et al., (2010), Neffe and Abell, (2005).

In one embodiment of the invention, the inhibitor of calpain activity may be calpastatin or truncated forms of calpastatin such as CP1B, PCP1B, 7-mer-PCP1B, 11R-CS, CP1B-[4-23] as described in U.S. Pat. No. 6,015,787; U.S. Pat. No. 6,294,518; U.S. Pat. No. 6,867,186.

In one embodiment of the invention, the inhibitor of calpain activity may be a peptidomimetic calpain inhibitor, a non-peptide calpain inhibitor such as disclosed in Donkor, (2011), and in the international patent application publication WO92/11850.

In one embodiment of the invention, the inhibitor of calpain activity is selected from the group consisting of SJA-6017, BDA-410, SNJ-1757, SNJ-1945, A-705253, MDL-28170, SC488, NS-398, SC-560, AK275, E64, calpeptin, calpastatin, acetyl-calpastatin, leupeptin, AK295, AK275, N-acetyl-leucyl-leucylmethional (ALLM or calpain inhibitor II), N-acetyl-leucylleucyl-norleucinal (ALLN or calpain inhibitor 1), calpain inhibitor III (carbobenzoxy-valyl-phenylalanal; Z-Val-PheCHO), calpain inhibitor IV (Z-LLY-FMK; Z-LLY-CH.sub.2 F where Z=benzyloxycarbonyl), calpain inhibitor V (MuVal-HPh-FMK where Mu is morphlinoureidyl and Hph is homophenylalanyl), mimetics thereof and compounds disclosed in U.S. Pat. No. 12,921,366; U.S. Pat. Nos. 5,716,980; 5,714,471; 5,693,617; 5,691,368; 5,679, 680; 5,663,294,5,661,150; 5,658,906; 5,654,146; 5,639,783; 5,635,178; 5,629,165; 5,622,981; 5,622,967; 5,621,101; 5,554,767; 5,550,108; 5,541,290; 5,506,243; 5,498,728; 5,498,616; 5,461,146; 5,444,042; 5,424,325; 5,422,359; 5,416,117; 5,395,958; 5,340,922; 5,336,783; 5,328,909; 5,135,916.

In one embodiment of the invention, the inhibitor of calpain activity is selected from synthetic calpain inhibitors such as PD-150606 ((2Z)-3-(4-iodophenyl)-2-mercapto-2-Propenoic acid, 3-(4-iodophenyl)-2-mercapto-(Z)-2-propenoic acid) and alpha-mercaptoacrylic acid derivatives such as disclosed in Wang et al., 1996.

In another embodiment the inhibitor of calpain activity is an aptamer. Aptamers are a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S.D., 1999. Peptide aptamers consists of a conformationally constrained antibody variable region displayed by a platform protein, such as E. coli Thioredoxin A that are selected from combinatorial libraries by two hybrid methods (Colas et al., 1996). Then after raising aptamers directed against calpains as above described, the skilled man in the art can easily select those inhibiting calpain.

In one embodiment the calpain inhibitor compound is an inhibitor of calpain expression.

Inhibitors of calpain expression for use in the present invention may be based on antisense oligonucleotide constructs. Anti-sense oligonucleotides, including anti-sense RNA molecules and anti-sense DNA molecules, would act to directly block the translation of calpain mRNA by binding thereto and thus preventing protein translation or increasing mRNA degradation, thus decreasing the level of calpain proteins, and thus activity, in a cell. For example, antisense oligonucleotides of at least about 15 bases and complementary to unique regions of the mRNA transcript sequence encoding calpain can be synthesized, e.g., by conventional phosphodiester techniques and administered by e.g., intravenous injection or infusion. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of calpain expression for use in the present invention. Calpain gene expression can be reduced by contacting the subject or cell with a small double stranded RNA (dsRNA), or a vector or construct causing the production of a small double stranded RNA, such that calpain expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of calpain expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of calpain mRNA sequences are thereby useful within the scope of the present invention. Specific ribozyme cleavage sites within any potential RNA target are initially identified by scanning the target molecule for ribozyme cleavage sites, which typically include the following sequences, GUA, GuU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of calpain expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing calpain. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Cliffton, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In one embodiment, the present invention relates to a method for preventing aging-related lesions in a subject in need thereof comprising the step of administering to said subject a calpain inhibitor compound.

In one embodiment, the present invention relates to a method for preventing cutaneous aging-related lesions in a subject in need thereof comprising the step of administering to said subject a calpain inhibitor compound.

In one embodiment, the present invention relates to a method for preventing hair aging-related lesions in a subject in need thereof comprising the step of administering to said subject a calpain inhibitor compound.

In one embodiment, the present invention relates to a method for preventing vascular aging-related lesions in a subject in need thereof comprising the step of administering to said subject a calpain inhibitor compound.

### Pharmaceutical Compositions

The calpain inhibitor compound of the invention may be used or prepared in a pharmaceutical composition.

In one embodiment, the invention relates to a pharmaceutical composition comprising the calpain inhibitor compound of the invention and a pharmaceutical acceptable carrier for use in a method for preventing aging related lesions in a subject in need thereof.

Typically, the calpain inhibitor compound of the invention may be combined with pharmaceutically acceptable excipients, and optionally sustained-release matrices, such as biodegradable polymers, to form therapeutic compositions.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The calpain inhibition compound of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

When the calpain inhibitor compound is intended for the prevention of cutaneous aging-related lesions, it may be desirable to it in admixture with a topical pharmaceutically or cosmetically acceptable carrier. The topical pharmaceutically acceptable carrier is any substantially nontoxic carrier conventionally usable for topical administration of pharmaceuticals in which the active ingredient of the invention will remain stable and bioavailable when applied directly to skin or corneal surfaces. For example, carriers such as those known in the art effective for penetrating the keratin layer of the skin into the stratum comeum may be useful in delivering the active ingredient of the invention to the area of interest. Such carriers include liposomes. Active ingredient of the invention can be dispersed or emulsified in a medium in a conventional manner to form a liquid preparation or mixed with a semi-solid (gel) or solid carrier to form a paste, powder, ointment, cream, lotion or the like.

Because dermatologic conditions to be treated may be visible, the topical carrier can also be a topical cosmetically acceptable carrier. The topical cosmetically acceptable carrier will be any substantially non-toxic carrier conventionally usable for topical administration of cosmetics in which the calpain inhibitor compound will remain stable and bioavailable when applied directly to the skin surface. Suitable cosmetically acceptable carriers are known to those of skill in the art and include, but are not limited to, cosmetically acceptable liquids, creams, oils, lotions, ointments, gels, or solids, such as conventional cosmetic night creams, foundation creams, suntan lotions, sunscreens, hand lotions, make-up and make-up bases, masks and the like. Topical cosmetically acceptable carriers may be similar or identical in nature to the above described topical pharmaceutically acceptable carriers. The compositions can contain other ingredients conventional in cosmetics including perfumes, estrogen, vitamins A, C or E, alpha-hydroxy or alpha-keto acids such as pyruvic, lactic or glycolic acids, lanolin, vaseline, aloe vera, methyl or propyl paraben, pigments and the like.

It may be desirable to have a delivery system that controls the release of the calpain inhibitor compound to the skin and adheres to or maintains itself on the skin for an extended period of time to increase the contact time of the calpain inhibitor compound on the skin. Sustained or delayed release of calpain inhibitor compound provides a more efficient administration resulting in less frequent and/or decreased dosage of calpain inhibitor compound and better patient compliance. Examples of suitable carriers for sustained or delayed release in a moist environment include gelatin, gum arabic, xanthane polymers. Pharmaceutical carriers capable of releasing the calpain inhibitor compound when exposed to any oily, fatty, waxy, or moist environment on the area being treated, include thermoplastic or flexible thermoset resin or elastomer including thermoplastic resins such as polyvinyl halides, polyvinyl esters, polyvinylidene halides and halogenated polyolefins, elastomers such as brasiliensis, polydienes, and halogenated natural and synthetic rubbers, and flexible thermoset resins such as polyurethanes, epoxy resins and the like. Controlled delivery systems are described, for example, in U.S. Pat. No. 5,427,778 which provides gel formulations and viscous solutions for delivery of the calpain inhibitor compound to a skin site. Gels have the advantages of having a high water content to keep the skin moist, the ability to absorb skin exudate, easy application and easy removal by washing. Preferably, the sustained or delayed release carrier is a gel, liposome, microsponge or microsphere.

### Screening Method

In a further aspect, the present invention relates to a method of screening a candidate compound for use as a drug for the prevention of aging-related lesions in a subject in need thereof, wherein the method comprises the steps of: i) providing candidate compounds and ii) selecting candidate compounds that blocks the action of calpain by inhibiting calpain activity or calpain expression, or by enhancing calpastatin activity or calpastatin expression.

In a further aspect, the present invention relates to a method of screening a candidate compound for use as a drug for the prevention of aging-related lesions in a subject in need thereof, wherein the method comprises the steps of:

(i) providing a calpain, providing a cell, tissue sample or organism expressing the calpain,

(ii) providing a candidate compound such as small organic molecule, intra-antibodies, aptamers, peptide or polypeptide,

(iii) measuring the activity of the calpain,

(iv) and selecting positively candidate compounds that blocks the action of calpain, inhibits calpain expression, enhances calpastatin activity or enhances calpastatin expression.

Methods for measuring the activity of the calpain are well known in the art. For example, measuring the calpain activity involves measuring the calpain inhibitory activity (Ki and IC₅₀ values), measuring Ca²⁺ transport on keratinocytes, podocytes, or cell lines expressing calpain or detection of cleavage of calpain substrates Ac-LLY-AFC, Suc-LLVY-AMC or BOC-LM-CMAC, or measuring spectrin breakdown in the presence or absence of the candidate compound.

Tests and assays for screening and determining whether a candidate compound is a calpain inhibitor compound are well known in the art (Niapour and Berger, 2007; Liu et al., 2012). In vitro and in vivo assays may be used to assess the potency and selectivity of the candidate compounds to reduce calpain activity.

Activities of the candidate compounds, their ability to bind calpain and their ability to inhibit calpain activity may be tested using isolated keratinocytes or podocytes expressing calpain, or cell lines expressing human calpain.

Cells, keratinocytes or podocytes expressing cathepsins such as cathepsin B, L or K but not expressing calpain may be used to assess selectivity of the candidate compounds.

The invention will be further illustrated by the following figures and examples. However, these examples and figures should not be interpreted in any way as limiting the scope of the present invention.

### FIGURES

FIG. 1: Specific calpain inhibition protects against age-related cardiovascular lesions. Aorta Wall surface μm².

FIG. 2: Specific calpain inhibition protects against telomere shortening. Telomeres length/quantitative PCR in kidney cortex.

FIG. 3: Telomere shortening with aging in control mice (WT) and transgenic mice (calpast). *=p<0.05, WT 2 years vs WT 2 months.

### EXAMPLES

### Example 1

Specific calpain inhibition protects arteries, kidneys, and skin against aging-related lesions: novel systemic or topic therapeutics against aging.

Material & Methods:

In Vivo:

In order to investigate long-term effect of calpains inhibition, inventors used mice overexpressing calpastatin, an original model developed in the UMR_S 702 laboratory. Calpain inhibition in this model is totally specific.

1. Twenty C57B16 female mice 2-years old (10 transgenic for calpastatin and 10 controls) bred in a specific pathogen free zone have been sacrificed in november 2010. Urine and blood have been collected. Main organs including kidneys, heart, brain, eyes, aorta have been either snap frozen, fixed with PFA/sucrose and frozen or fixed with AFA and embedded in paraffin. Morphological and immunohistochemical analysis have been performed with a special focus on inflammation, fibrosis (sirius red, collagens). Kidney, heart, liver, white blood cell and aorta samples have been isolated for studying mRNA and protein expression and/or telomere length.

2. Twenty C57B16 2-months old female mice (10 transgenic for calpastatin and 10 controls) have been sacrificed as controls in comparison to 2-years-old mice.

3. In view of the first results, 10 additional female mice transgenic for calpastatin and 10 controls have been bred in a pathogen specific free zone during one year. Their arterial blood pressure has been measured in a conventional breeding zone during 2 weeks and by invasive hemodynamic methods. The inventors also measured glomerular filtration rate and renal blood flow in both groups.

In Vitro

Inventors first focused their work on somatotropic axis and particularly IGF-1 receptor role in calpain activation. Actually, IGF-1 has been shown to play an important role in aging process (8). Calpain activity assays developed in the laboratory have been performed in several cell lines expressing IGF-1 receptor (podocyte lines, splenocytes, mesangial cells) after exposure to IGF-1.

Results:

1. Specific Calpain Inhibition Protects Against Age-Related Cardiovascular Lesions:

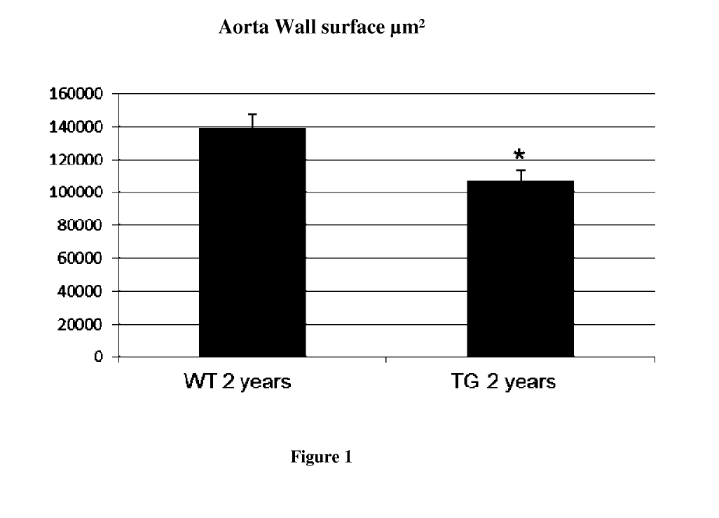

Ca1pastatin transgenic animals are protected against aging-related vascular remodelling. Thoracic aorta diameter and media thickness was similar in 2 months-old transgenic and control animals. Control animals sacrificed at 2 years had a significantly increased aortic diameter and media thickness, as compared to young ones. By contrast, 2 years-old transgenic mice were protected against aortic excentric and concentric remodelling (FIG. 1). A similar protection has been observed in smaller arteries, interlobar arteries in kidneys whose diameter does not increase with aging in transgenic animals.

These results cannot be explained by hemodynamic changes since blood pressure, as assessed by both non-invasive and invasive methods, was similar in the two groups of one-year old mice. Moreover, glomerular filtration rate and renal blood flow, as assessed by invasive methods, were similar in the 2 groups.

2. Specific Calpain Inhibition Protects Against Glomerulosclerosis and Age-Related Kidney Interstitial Fibrosis:

Histological aspect of kidneys was similar in young wild type and calpastatin transgenic mice. At 2 years, all control mice (10/10) exhibited enlarged glomeruli affected by sclerosis whereas most of transgenic mice (8/10) had a conserved kidney architecture. Interstitial fibrosis assessed by Sirius Red staining in polarized light revealed that transgenic mice had significantly less interstitial fibrosis than controls.

3. Specific Calpain Inhibition Protects Skin and Hair from Aging

All control animals bred in a specific pathogen free zone and sacrificed at 2 years (n=10) had the typical aspect of C57b16 “old” mice with depilated and brown fur and skin lesions whereas all transgenic mice (n=10) had a black fur without skin lesions, exactly similar to 2 month old C57b16 mice.

4. Specific Calpain Inhibition Protects Against Telomere Shortening

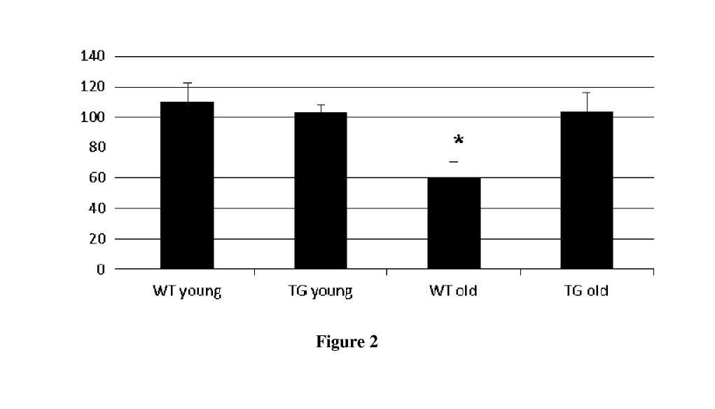

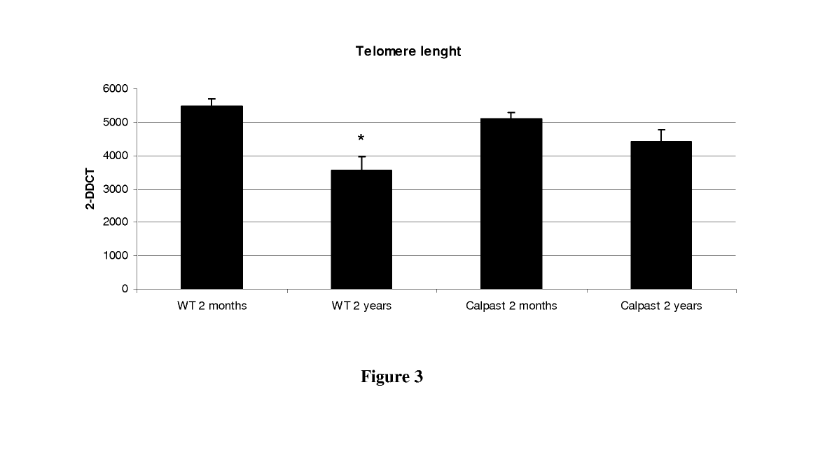

Telomere shortening is a specific feature of cell senescence. Inventors observed a significant shortening of telomeres in the control group in skin and kidney cortex with aging whereas telomere length was preserved in skin and kidney cortex of calpastatin transgenic animals (FIGS. 2 and 3).

5. In Vitro

Inventors evidenced that IGF-1 was able to increase calpain activity in a dose- and time-dependent manner in cell lines, including immortalized podocytes, providing evidence that somatotropic axis, which is implied in aging process, results in calpain activation.

Conclusion:

Inventors evidenced for the first time that specific calpain inhibition protects against telomere shortening and aging lesions in various tissues including arteries and skin. Although calpain activity is known to increase in tissues with aging and may play a role in Alzheimer disease, there was no data indicating that specific calpain inhibition was able to prevent vascular aging process. Unexpectedly, inventors observed that skin of mice with decreased calpain activity (calpastatin transgenic) remained similar to young mice skin. Thus, specific calpain inhibition by oral, intravenous, intramuscular or topic administration of specific inhibitors (natural or synthetic) may protect arteries, kidneys or skin against aging lesions. The use of topic calpain inhibitors may prevent skin aging. In the same way, drugs enhancing calpastatin expression may protect against tissue aging.

### Example 2

Background:

Aging is characterized by declines and lesions in various organs including skin, hair and fur in animals. Mice are popular mammalian species for aging studies and skin lesions related to aging have been characterized in C57B1/6J mice (9). These mice exhibit alopecia and fur mites, fur colour modifications, dermatitis, scarring and barbering with aging. At the tissular and cellular level, histopathological modifications such as lipofuscin deposits have been associated to aging in various tissues, including skin (10). The most striking discovery in the field of aging during the past decades is that telomeres shorten with aging. Telomeres are DNA sequences with a structure that protects tip chromosomes from erosion. Telomere length measure is the hallmark feature of senescence (11-12).

The inventors assessed skin senescence in 2-months and 2-years old female mice at three levels: (i) Phenotypic evidence, (ii) Histopathological modifications, especially lipofuscin deposits, and (iii) Measure of telomere length in skin.

Material and Methods

1. Visual Assessment of Skin and Fur Alterations:

Two independent investigators assessed the existence of a phenotypic evidence for skin and/or fur alteration, defined by greying/brown discoloration of the fur and sparse depilation. 40 mice were examined (2 months and 2 years old control and transgenic calpastatin over-expressing female mice, n=10/group). Transgenic mice have been created in the laboratory as previously described (13) 2. Histological Examination

Skin pieces (1 cm²) were harvested from 40 mice at the time of sacrifice (2 months and 2 years old, controls and transgenic calpastatin over-expressing mice, n=10/group). Skin pieces were snap frozen and 4 μm sections were performed and then fixed in acetone. Tissues were analyzed with a fluorescence microscope to assess autofluorescence of lipofuscin deposits and by histochemistry (Periodic Acid Schiff coloration) to assess the typical brown deposits of lipofuscin in skin. The inventors have chosen a semi-quantitative approach to evaluate lipofuscin deposits since they are irragularly accumulated in dermis and quantitative measures might be biased. Two independent investigators assessed whether deposits were present and/or abundant.

3. Telomere Length Measure by Quantitative PCR:

Genomic DNA Extraction from Skin:

Skin pieces were harvested from 40 mice at the time of sacrifice (2-months and 2-years old, control and transgenic calpastatin overexpressing mice, n=10/group). Twenty-five mg of skin tissues was incubated overnight in ATL lysis buffer supplemented with proteinase K at 56° C. DNA was extracted by using DNeasy Mini spin columns (QIAGEN, Hilden, Germany). DNA quality was assessed by Nanodrop 1000 (Thermo Fisher Scientific, Wilmington, USA): absorbance ratio A260 nm/A280 nm should range from 1.7 to 2.

Telomere Length Quantitative PCR

Telomere length was measured by quantitative polymerase chain reaction (PCR) according to the protocol of Cawthon et al adapted for mice tissues (14-16). Telomere specific repetitive sequences were amplified in a thermocycler (LightCycler 480, Roche Diagnostics) as follows: 95° C. for 5 min followed by 35 cycles at 95° C. for 15 s, 52° C. for 20 s, and 72° C. for 15 s by using SYBR Green (Roche Diagnostics) and 2.5 pmol specific primers for mouse telomeres. 36B4 was the housekeeping genes used for normalization, amplified in a thermocycler as follows: 95° C. for 5 min followed by 35 cycles at 95° C. for 15 s, 56° C. for 20 s, and 72° C. for 15 s by using SYBR Green (Roche Diagnostics) and 2.5 pmol specific primers for 36B4. Results are expressed as 2^{-ΔΔCt}.

Results:

Three lines of evidence advocate the role of calpain inhibition as protecting against aging-related lesions in skin:

1. Phenotypic Evidence

Calpastatin-overexpressing with reduced calpain activity, and control mice with the same genetic background (C57B1/6), were sacrificed at 2 months and 2 years. 2-month old mice had the typical black fur of C57B1/6 mice. At 2 years, all of the 10 control animals exhibited discoloration of the fur and sparse depilation whereas none of the calpastatin-overexpressing mice had alterations of fur (n=10/group, p=5×10⁻⁵, Pearson's Chi-squared test with Yates' continuity correction).

2. Autofluorescence of Skin Lipofuscin Deposits and Histochemistry

No lipofuscin deposits were observed in 2-month old mice from both groups. Lipofuscin deposits observed by autofluorescence were abundant in most of 2-years old control mice but rare in 2-years old calpastatin transgenic animals. Histochemistry confirmed autofluorescence data and was considered as more adapted to assess precisely the abundance of lipofuscin deposits. Independent investigators assessed that lipofuscin deposits were significant in 70% of 2-years old control mice control and 10% of 2-years old calpastatin transgenic mice (n=10/group p=0.02, Fisher's exact test, two sided)

3. Telomere Shortening in Skin

Telomere shortening was significant in control mice between 2 months and 2 years (n=10/group, p=3.10⁻⁴, bilateral student t test) but not significant between 2-months and 2-years old transgenic calpastatin mice, proving therefore that calpastatin over-expression protects against skin aging (FIG. 3).

### Example 2

Specific Calpain Inhibition Protects from Aging

The inventors evidenced that IGF-1 was able to increase calpain activity in a dose- and time-dependent manner in cell lines, including immortalized podocytes, providing evidence that somatotropic axis, which is implied in aging process, results in calpain activation.

Inventors evidenced that calpain inhibition by transgenic overexpression of calpastatin or by the synthetic calpain inhibitor PD-150606 blunted partly but significantly the mitogenic effect of IGF-1 on mouse smooth muscle cells in vitro. These results evidence that pharmacological and genetic blockade of calpains as well inhibit somatotropic axis signalling in mammal cells.

In addition, the inventors transfected HEK cells by a FOXO reporter measuring the activity of the Pl3kinase/Akt pathway downstream of IGF-1 receptor. They evidenced that pharmacological blockade of calpains by PD-150606 inhibited significantly PI3kinase/Akt pathway in transfected cells exposed to IGF-1.

Taken together, in vitro experiments evidence that synthetic compounds inhibiting calpain activity (i.e. calpain inhibitors) blunt somatotropic axis signalling, a major pathway involved in aging processes. Accordingly, specific calpain inhibition by genetic or pharmacological ways protects tissues against aging.

### REFERENCES

Throughout this application, various references describe the state of the art to which this invention pertains. The disclosures of these references are hereby incorporated by reference into the present disclosure.

1. Letavernier E, Perez J, Bellocq A, Mesnard L, de Castro Keller A, Haymann J P, Baud L. Targeting the Calpain/Calpastatin System as a New Strategy to Prevent Cardiovascular Remodeling in Angiotensin II-Induced Hypertension. Circ Res. 2008 Mar. 28; 102(6):720-8.

2. Goll D E, Thompson VF, Li H, Wei W, Cong J. The calpain system. Physiol Rev 2003; 83(3):731-801.

3. Saito K, Elce J S, Hamos J E, Nixon R A. Widespread activation of calcium-activated neutral proteinase (calpain) in the brain in Alzheimer disease: a potential molecular basis for neuronal degeneration. Proc Natl Acad Sci USA 1993; 90(7):2628-32.

4. Lee M S, Kwon Y T, Li M, Peng J, Friedlander R M, Tsai L H. Neurotoxicity induces cleavage of p35 to p25 by calpain. Nature 2000; 405(6784):360-4.

5. Trinchese F, Fa' M, Liu S, Zhang H, Hidalgo A, Schmidt S D et al. Inhibition of calpains improves memory and synaptic transmission in a mouse model of Alzheimer disease. J Clin Invest. 2008 August; 118(8):2796-807.

6. Nakamura Y, Fukiage C, Shih M, Ma H, David L L, Azuma M, et al. Contribution of calpain Lp82-induced proteolysis to experimental cataractogenesis in mice. Invest Ophthalmol Vis Sci 2000; 41(6):1460-6.

7. Jiang L, Wang M, Zhang J, Monticone R E, Telljohann R, Spinetti G, et al. Increased aortic calpain-1 activity mediates age-associated angiotensin II signaling of vascular smooth muscle cells. PLoS ONE 2008; 3(5):e2231.

8. Holzenberger M, Dupont J, Ducos B, Leneuve P, Géloën A, Even P C, Cervera P, Le Bouc Y. IGF-1 receptor regulates lifespan and resistance to oxidative stress in mice. Nature. 2003 Jan. 9; 421(6919):182-7.

9. Pettan-Brewer C, Treuting P M. Practical pathology of aging mice. Pathobiol Aging Age Relat Dis. 2011; 1. doi: 10.3402/pba.vli0.7202.

10. Jung T, Holm A, Catalgol B, Grune T. Age-related differences in oxidative protein-damage in young and senescent fibroblasts. Arch Biochem Biophys. 2009 Mar. 1; 483(1):127-35.

11. de Jesus B B, Blasco M A. Assessing cell and organ senescence biomarkers. Circ Res. 2012 Jun. 22; 111(1):97-109.

12. Buckingham E M, Klingelhutz A J. The role of telomeres in the ageing of human skin. Exp Dermatol. 2011 April; 20(4):297-302.

13. Peltier J, Bellocq A, Perez J, Doublier S, Dubois YC, Haymann J P, Camussi G, Baud L.

Calpain activation and secretion promote glomerular injury in experimentalglomerulonephritis: evidence from calpastatin-transgenic mice.J Am Soc Nephrol. 2006 December; 17(12):3415-23.

14. Callicott R J, Womack J E. Real-time PCR assay for measurement of mouse telomeres. Comp Med. 2006 February; 56(1):17-22.

15. Cawthon R M. Telomere measurement by quantitative PCR. Nucleic Acids Res. 2002 May 15; 30(10):e47.

16. Cawthon R M. Telomere length measurement by a novel monochrome multiplex quantitative PCR method. Nucleic Acids Res. 2009 February; 37(3):e21.

17. Niapour M, Berger SNiapour M, Berger S. Flow cytometric measurement of calpain activity in living cells. Cytometry A. 2007 July; 71(7):475-85.

18. Liu D, Yan Z, Minshall R D, Schwartz D E, Chen Y, Hu G. Activation of calpains mediates early lung neutrophilic inflammation in ventilator-induced lung injury. Am J Physiol Lung Cell Mol Physiol. 2012 Feb. 15; 302(4):L370-9. Epub 2011 Dec. 2.

19. Wang K K, Posner A, Raser K J, Buroker-Kilgore M, Nath R, Hajimohammadreza I, Probert A W, Marcoux F W, Lunney E A, Hays S J, Yuen P-W. Alpha-mercaptoacrylic acid derivatives as novel selective calpain inhibitors. Adv Exp Med Biol. 1996; 389:95-101.

1. A method for preventing aging-related lesions in a subject in need thereof comprising the step of administering to said subject a calpain inhibitor compound.

2. The method of claim 1, wherein said aging-related lesions are cutaneous aging-related lesions.

3. The method of claim 1, wherein said aging-related lesions are hair aging-related lesions.

4. The method of claim 1, wherein said aging-related lesions are vascular aging-related lesions.

5. The method according to claim 1, wherein said calpain inhibitor compound is selected from the group consisting of an inhibitor of calpain activity, a calpain expression inhibitor, a calpastatin activator and a calpastatin expression activator.

6. The method according to claim 5 wherein said inhibitor of calpain activity is calpastatin or a truncated form of calpastatin.

7. The method according to claim 2, wherein said calpain inhibitor compound is selected from the group consisting of an inhibitor of calpain activity, a calpain expression inhibitor, a calpastatin activator and a calpastatin expression activator.

8. The method according to claim 7 wherein said inhibitor of calpain activity is calpastatin or a truncated form of calpastatin.

9. The method according to claim 3 wherein said calpain inhibitor compound is selected from the group consisting of an inhibitor of calpain activity, a calpain expression inhibitor, a calpastatin activator and a calpastatin expression activator.

10. The method according to claim 9 wherein said inhibitor of calpain activity is calpastatin or a truncated form of calpastatin.

11. The method according to claim 4, wherein said calpain inhibitor compound is selected from the group consisting of an inhibitor of calpain activity, a calpain expression inhibitor, a calpastatin activator and a calpastatin expression activator.

12. The method according to claim 11 wherein said inhibitor of calpain activity is calpastatin or a truncated form of calpastatin.
